(19) United States
(12) Patent Application Publication
(10) Pub. No.: US 2024/0310370 A1
(43) Pub. Date: Sep. 19, 2024

(54) Method For Concentration And Avidity Detection Of Antibodies Against Immune System Related Diseases Measured With Evanescent Field Based Biosensors Using A Continuous Gradient Of Ligand Densities

(71) Applicant: VYSENS B.V., Hengelo (NL)

(72) Inventor: Richard B.M. SCHASFOORT, Haaksbergen (NL)

(73) Assignee: VYSENS B.V., Hengelo (NL)

(21) Appl. No.: 18/647,791

(22) Filed: Apr. 26, 2024

(30) Foreign Application Priority Data
Nov. 4, 2022 (WO) PCT/EP2022/080871

(51) Int. Cl. G01N 33/543; G01N 33/68

(52) U.S. Cl. CPC G01N 33/54373; G01N 33/6854; G01N 2333/165; G01N 2800/26; G01N 2800/52; G01N 2800/56

(57) ABSTRACT

The core of this patent application is about the invention of a plug & play method and device for concentration (titer) and avidity parameters using continuous gradients of ligand densities applied on the biosensor surface. In a previous study, humoral antibody responses and strength of binding against specific SARS-CoV-2 proteins was investigated. Surface Plasmon Resonance imaging (SPRi) was used to determine the strength of binding of IgG, IgM and IgA against the Receptor Binding Domain and Nucleocapsid (RBD/NCP) of SARS-CoV-2 in sera of 119 COVID-19 patients. Interestingly, in contrast to the titer of antibodies, the binding strength (off-rate increased) went down with increasing disease severity. The invention is a measure of concentration and affinity/avidity parameters in a single experiment in a plug & play manner. The ligand density is a critical parameter with a huge effect on the value of the off-rate. A factor 10 mismatch can easily be obtained. Besides off-rate also the on-rate at a fixed ligand density should be measured, but then the effective concentration should be known too. The core of the invention is to apply a continuous gradient on the sensor surface for determining the titer and avidity of the antibodies and measure the avidity parameter at constant but low Rₘₐₓ value and the concentration of the antibodies at high Rₘₐₓ values. In this way, generated antibodies to infectious disease can be determined for each patient in particular for determining and prediction of COVID19 severity. A double gradient method can be applied to measure on two targets simultaneously.

### CROSS REFERENCE TO RELATED APPLICATIONS

This application is the US national application of PCT/EP2022/080871, filed 4 Nov. 2022, which claims benefit of and priority to U.S. Provisional Patent Application No. 63/275,581, filed 4 Nov. 2021, where permissible incorporated by reference in its entirety.

### BACKGROUND

### Field of the Invention

The present invention relates to a method for assessing concentration and affinity/avidity parameters of label free antibodies against infectious diseases using gradients of ligand densities detected by Surface Plasmon Resonance imaging. The quantity at high ligand density and quality at low but fixed ligand density of generated antibodies to infectious disease can be determined for each patient in particular for determining and prediction of COVID-19 severity.

### Description of Related Art

After almost two years from the beginning of the COVID-19 pandemic it is still unknown how the humoral response affects disease progression. We investigated humoral antibody responses against specific SARS-CoV2 proteins, their strength of and their relationship with COVID severity and clinical information. For this purpose, a new method has been developed for plug & play analysis of concentration and avidity parameters label free using gradients of ligand densities detected by Surface Plasmon Resonance imaging. We measured specific antibodies of isotypes IgM, IgG and IgA as well as their binding strength against the SARS-CoV2 antigens RBD/NCP, NCP, S1 and S1S2 in sera of 76 COVID-19 patients using Surface Plasmon Resonance imaging (SPRi). We observed a positive association between disease severity and IgG antibody titers against all SARS-CoV2 proteins, and additionally for IgM and IgA antibodies directed against RBD/NCP. Interestingly, in contrast to the titer of antibodies, the binding strength went down with increasing disease severity. Within the critically ill patient group, a positive association with pulmonary embolism, d-dimer and antibody titers was observed. We hypothesize that in critically ill patients, antibody production and/or maturation may be ineffective. This approach may aid in predicting COVID-19 severity by measuring the quantity and quality of the antibody responses using label-free bio sensing techniques. The invention discloses a method to measure concentration and affinity parameters in a single experiment in a plug & play mode in approximately 10 minutes. During the course of the disease, the IgG levels and strength of binding increased while generally the IgM and IgA levels went down. Recovered patients all show high strength of binding of the IgG type to the RBD/NCP protein. The anti-RBD/NCP immune globulins SPRi assay provides additional insights into the immune status of patients recovering from COVID-19 and can be applied for the assessment of the immune reaction of healthy individuals in vaccination programmes. It turned out that at least two SARS-CoV-2 proteins should be applied for measuring the polyclonal immune profile; RBD and NCP. The strength of binding in terms of off-rate values correlated with the prediction of COVID-19 severity. The invention applies dissociation of the bound antibodies for all the patients simultaneously not only on Spike proteins (e.g. the Receptor Binding Domain (RBD/NCP), S1, S2, S1S2 and sub-epitopes) but also on the highly immunogenic nucleocapsid protein (NCP) and epitopes from NCP. Generally, by applying strong immunogenic epitopes of the virus, bacterium or other foreign invading species in this way the quantity and quality of generated antibodies to infectious disease can be determined for each patient—in particular for determining and prediction of COVID19 severity. This parameter of strength of binding for at least two immunogenic proteins and the ratio between off-rates is for the first time proven as an important indicator of COVID-19 severity. However, the off-rate could be sufficient to determine the binding strength but it is better to get a reliable indication of the equilibrium dissociation constant by measuring besides the off-rate also the on-rate. A new double gradient method was applied to measure on- and off-rates quantitatively and accurately. The method is described herein.

The coronavirus 2019 (COVID-19) pandemic has disrupted global society, critically stressed healthcare systems, and resulted in a relatively high mortality and morbidity with continued high need for patient care. Even though one and a half year of intensive international scientific effort has provided a large amount of information, many questions remain about the underlying pathology, distinctive patient factors determining severity and on the protective or damaging role of humoral immune system.

In fact, there is emerging evidence of a potential deleterious role of the humoral response in the severity of the disease. Therefore, there appears to be a delicate balance of a protective effect and hyper-reaction of the immune system leading to organ/tissue damage and potential death.

Yet, the factors determining the balance between disease attenuation and disease amplification are not well understood. For example, most papers focus on single viral proteins (in particular spike (S) or nucleocapsid (NCP)). Therefore, there is limited information on the dynamics of the antibody response towards specific viral targets (e.g. receptor binding domain (RBD, S1, S2 or NCP) and the ratio of the response to these viral proteins. Moreover, several studies contradict each other regarding the longitudinal trends in antibody production and titer in mild versus severe disease. These limitations and contradictory results are partly caused by the lack of consistency in comparison groups, study design, the heterogeneity of assays that were used and by indirect antibody measurements. Antibody measurements are typically performed using ELISA or related immunoassays. In addition to relative long assay times, testing of IgM, IgG, and IgA isotopes require individual assays for either techniques. Furthermore, standard immunoassays provide only indirect information on antibody kinetics and affinities.

An attractive alternative is surface plasmon resonance imaging (SPRi). In previous studies, we have demonstrated a high throughput SPRi assay for the quantitative measurement of IgM, IgG and IgA antibodies and their apparent polyclonal affinity in the sera of COVID-19 patients in one single experiment with a run time of less than 30 minutes. This method is ideally suitable for measuring concentrations of antibodies in patients as well as determining their strength of binding using off-rate detection.

The immune reaction to corona viruses generally provides immunity via neutralizing antibodies in the event of a second exposure to the virus. However, the ability for the generation of neutralizing antibodies only is not sufficient to predict COVID-19 severity. At least two different highly immunogenic proteins of the virus should be used to measure the immune response to these proteins. Serological antibody testing is essential to get an indication whether or not an individual has been infected with SARS-CoV-2. The quality of the immune response is not only determined by the quantity of antibodies but also by the overall strength of binding of the pool of potential neutralizing antibodies that binds to the relevant immunogenic proteins of the corona virus. Recently it was shown that the most potent, highest affinity neutralizing antibodies were directed to the receptor-binding domain (RBD). This RBD of SARS-CoV-2 domain binds to the angiotensin converting enzyme2 (ACE2) receptor expressed by target cells. The affinity of the RBD of SARS-CoV and SARS-CoV-2 to the ACE2 receptor appeared to be approximately K_{D}˜10 nM as determined by surface plasmon resonance. Hence, the quality of the antibodies to SARS-CoV-2 should be more than a factor 6 better than antibodies to SARS-CoV in order to prevent the virus binding to its receptor. Generally, an antibody in that range will effectively block the virus monovalently. Polyclonal supporting antibodies preferably of high affinity (<nM) should be raised additionally to block the RBD of SARS-CoV-2 from binding to the ACE2 receptor and allow the removal of the virus via e.g. nucleocapsid domains. If these antibodies show less affinity then an overwhelming Fc reaction is present and the immune system will be over activated.

An attractive alternative for antibody detection is an evanescent field based photonic device as e.g. surface plasmon resonance imaging (SPRi) among other photonic sensors. SPRi is a label-free sensing technique that is highly sensitive enabling the quantitative and qualitative interaction between biomolecules, such as the interaction between antibodies and their respective antigens. More importantly, the strength of binding measured by the off-rate can be determined in a single assay to obtain an indication of the quality of the total polyclonal antibody response. The discrimination and ratio between at least two highly immunogenic proteins from SARS-CoV-2 predicts the severity of COVID-19 as disclosed herein.

### SUMMARY OF THE INVENTION

For getting data as described herein, we describe a high throughput Surface Plasmon Resonance imaging (SPRi) assay for the quantitative measurement of IgG, IgM and IgA antibodies binding to the RBD spike protein and the NucleoCapsid protein (NCP) and their apparent polyclonal affinity in sera of COVID-19 patients. If the concentration of specific antibodies is low, then the specific interaction interferes with the non-specific interaction, resulting in false positive results. However, we found that the strength of binding of a specific biomolecular interaction is often stronger than the non-specific interaction. Predicting, the severity of the disease by measuring the ratio of levels and affinity of at least two immunogenic proteins is the core of the invention. Additionally to this core of the invention, a simultaneous but partial elution of the antibodies bound to the specific antigen will reveal easily the difference between the off-rates of antibodies bound to the immunogenic proteins or antibodies bound non-specifically. This not only reveals the strength of binding of the three isotype antibodies (M, G, A) to at least two immunogenic proteins of the virus but also reduces the number of false-positives.

The core of the present invention is about a plug & play method for concentration (titer) and avidity parameters using continuous gradients of ligand densities applied on the biosensor surface. In a previous study humoral antibody responses and strength of binding against specific SARS-CoV2 proteins was investigated. Surface Plasmon Resonance imaging (SPRi) was used to determine the strength of binding of IgG, IgM and IgA against the Receptor Binding Domain and Nucleocapsid (RBD/NCP) of SARS-CoV-2 in sera of 119 COVID-19 patients. Interestingly, in contrast to the titer of antibodies, the binding strength (off-rate increased) went down with increasing disease severity. The invention provides a method to measure concentration and affinity/avidity parameters in a single experiment in a plug & play manner in e.g. 10 minutes. A well-known observation is that the ligand density is a critical parameter with a huge effect on the value of the off-rate. A factor 10 mismatch can easily be obtained. Besides off-rate also the on-rate at a fixed ligand density should be measured, but then the effective concentration should be known too. The core of the invention is to apply a continuous gradient on the sensor surface for determining the titer and avidity of the antibodies and measure the avidity parameter at constant but low Rₘₐₓ value and the concentration of the antibodies at high Rₘₐₓ values. In this way the quantity (at high ligand density) and quality (at low but fixed ligand density) of generated antibodies to infectious disease can be determined for each patient in particular for determining and prediction of COVID-19 severity. A double gradient method can be applied to measure on two targets simultaneously.

In one aspect, the invention provides a method for predicting infectious disease severity by the combination of specific antibody concentration and strength of binding of all antibodies isotypes from a body fluid sample taken from a patient, the method comprising the steps of: exposing the sample of the patient's body fluid having an infectious disease to an immunogenic antigen immobilized in a gradient on a label-free and real-time imaging biosensor; determining a concentration at high ligand density using an initial slope of a binding curve under mass transport limited conditions; and determining avidity parameters at low but fixed Rₘₐₓ conditions on a ligand density gradient, wherein the ratio of levels and affinity of at least two immunogenic proteins predicts the severity of the infectious disease in the patient.

In some embodiments, the label-free and real-time biosensor is based on any evanescent field optical phenomenon. In some embodiments, the evanescent field based biosensor is an optical device based on Surface Plasmon Resonance (SPR) imaging.

In some methods according to the invention, a controlled injection of a ligand creates a gradient in ligand density by differences in contact time using at least a single back and forth flow of the sample in the flow channel in contact with a sensor surface.

In some instances, fluidics are designed such that the sample can simultaneously address at least two channels or more in gradients for measuring the concentration and the association rate and dissociation rate simultaneously for at least a duplex measurement of the same sample.

Methods according to the invention may be utilized where the infectious disease is COVID-19. In some examples, the immobilized immunogenic antigens are at least a Receptor Binding Domain and Nucleocapsid (NCP) of SARS-CoV-2. In such examples, the binding constants of the Nucleocapsid antibodies and the binding constants of RBD may predict COVID-19 severity of infectious disease in the patient.

In some methods according to the invention, half a flow cell is applied for timely exposure of the ligand from inlet to outlet to create a gradient in ligand density of the first immunogenic protein by rotating the sensor 180 degrees to allow a next immobilization with the second protein in a timely exposure from inlet to outlet of the flow cell at the top section while the first gradient is still on the down section whereby the sensor is installed with the multiplex flow cell for measuring the strength of binding and isotypes of the bound antibodies for at least two immunogenic proteins simultaneously.

In some instances, measurement of the biomolecular interaction on the gradient for finding the fixed Rₘₐₓ value includes analysis of the on- and off rates using the sensorgrams with the fixed Rₘₐₓ value for all ligand gradients while simultaneously the concentration is measured at another location on the gradient. In such cases, the biomolecular interaction on the gradients may be simultaneously exposed to concatenated injections of anti-isotype antibodies.

Some methods according to the invention may be utilized to establish the strength of binding of isotypes of antibodies using SPR imaging and to reduce the number of false positives, characterized by a print of gradients of ligands exposed to patient samples.

In another aspect the invention provides a device comprising: an SPR imager; and a line printer for creating a gradient in a track; wherein parts of the printed line or track is exposed to the patient sample.

In some embodiments of device according to the invention, a single channel is applied on printed tracks and the sensor prism is repositioned to cover a part of the tracks.

In other embodiments of device according to the invention, a single channel is applied on printed tracks and the flow cell is repositioned to cover a part of the tracks.

In still further embodiments, crisscross tracks are applied to create ligand density gradients by either replacing the sensor prism or the flow cell position.

The invention is described below in further detail by way of example only and with reference to certain preferred embodiments provided for illustrative purposes.

### BRIEF DESCRIPTION OF THE FIGURES

FIG. 1 Justification for a gradient plug & play kinetic parameter analysis. Shown is a current standard procedure having a measurement time of approximately 1 hour (Panel A) compared with the measurement time of approximately 5 minutes in the present invention (Panel B).

FIG. 2 Representative image creating a gradient on a sensor prism using contact time differences. (Panel A) The ligand is immobilized more on the right than on the left. The ligand will flow from one direction and back to enable contact time differences. (Panel B) contains representative images A-B-C showing the contact time gradient in the flow cell.

FIG. 3 Representation of the three regimes in the gradient for measuring biomolecular interactions.

FIG. 4 Representative image for the generation of two gradients in opposite directions.

FIG. 5 Image representation of the holder for the sensor prism for printing a double gradient. (A) the sensor holder is shown with positioning cross (B) and bayonet closure (C).

FIG. 6 Principle of the anti-SARS-CoV-2 immune globulins isotypes of SPRi assay. The process of spotting the sera (A) to an RBD and NCP coupled surface in the MX96 SPRi instrument (B) resulted in a SPRi reflectivity image (C). In (D) the sensorgram is shown of injections of three antisera to determine the response of IgM, IgG and IgA antibodies. In panel (E) an overlay of the injections of the anti-IgM, IgA and IgG antibodies of a single spotted serum is presented for calculating the Rₘₐₓ values of the IgM, IgG and IgA binding, after zeroing, aligning the sensorgram.

FIG. 7 Infographics outlining the experimental design for using SPRi to characterize patient samples for the presence of SARS-CoV-2 antibody responses. A) IgM, IgG and IgA antibody response towards RBD, NCP, S1S2 and S2 proteins is measured sequentially using SPRi. First patient plasma is incubated on a specific protein coupled sensor, then anti-IgM, anti-IgG and anti-IgA are sequentially injected and association signal is measured in real-time. B) Affinity of patients' polyclonal antibody pools towards RBD, NCP, S1S2 and S2 proteins are determined. Patient plasma is injected on protein coupled sensor and interactions are measured in real-time. The k_{off} constant determines strength of binding and is determined in dissociation phase.

FIG. 8 The dissociation constant of the anti-RBD antibodies as function of the days after symptoms onset. The middle trend line is the overall line of all measured samples. The other lines connect four longitudinal samples in duplicate. This principle is the classical way of measuring the total affinity.

FIG. 9 The strength of binding of anti-NCP antibodies is lower (dissociation rate higher) than the anti-RBD antibodies in moderate and critical patients. Data obtained from Hendriks et al. The association and dissociation rate is measured with any kind of photonic device label free and in real time.

FIG. 10 Multiplex measurement of antibody responses to four antigens of SARS-CoV-2. A) Total immune response (IgM, IgG, IgA) SPRi measurement of COVID-19 positive sera on RBD/NCP antigen. B) IgG immune response SPRi measurement of NCP, S1S2 and S2 antigen. The boxplots represent the median, p25 and p75 values and the black dot the mean SPRi RU value. Comparability of groups was analyzed by Mann-Whitney U-test. A Bonferroni-Holm procedure was used to correct for multiple comparisons between groups (*p<0.05 and **p<0.01 vs mild group and #p<0.05 vs moderate group).

FIG. 11 Binding strength measurements of four antigens of SARS-CoV-2. The off-rate of the antibodies binding to the four antigens was determined for each severity group. The boxplots represent the median, p25 and p75 values and the black dot the mean SPRi RU value. Comparability of groups was analyzed by Mann-Whitney U-test. A Bonferroni-Holm procedure was used to correct for multiple comparisons between groups (*<0.05 and **<0.01 vs mild group and #p<0.05 and ##p<0.01 vs moderate group by Mann-Whitney U test).

FIG. 12 Binding strength and the correlation with gender. On the left are the boxplots representing the critical patients and on the right the moderate patients. As can be seen, there is a significant difference in the critical patients group for female and male for RBD (p=0.043). Others were non-significant. The boxplots represent the median, p25 and p75 values and the black dot the mean SPRi RU value (*p<0.05 by Mann-Whitney U test).

FIG. 13 A COVID-19 survivor or a COVID-19 deceased patient shows a different immune quality profile illustrated in this sketch, which is unique for each patient. It is still unclear why the human body generates such an enormously diverse polyclonal immune response to SARS-CoV-2 over the human population. So, the immune fingerprint in terms of antibody levels and strength of binding should be tested for critical parameters in order to predict COVID-19 severity.

### DETAILED DESCRIPTION OF INVENTION

This invention was supported in part by the ATTRACT Project funded by European Council (EC) under Grant 777222 and in part by the European Regional Development Funded under Grant 10.13039/5011000008530.

The study was performed in accordance with the guidelines for sharing of patient data of observational scientific research in emergency situations as issued by the Commission on Codes of Conduct of the Foundation Federation of Dutch Medical Scientific Societies (https://www.federa.org/federa-english).

As stated herein, we describe a high throughput Surface Plasmon Resonance imaging (SPRi) assay for the quantitative measurement of IgG, IgM and IgA antibodies binding to the RBD spike protein and the NucleoCapsid protein (NCP) and their apparent polyclonal affinity in sera of COVID-19 patients. If the concentrations of specific antibodies are low, then the specific interaction interferes with the non-specific interaction resulting in false positive results. However, we found that the strength of binding of a specific biomolecular interaction is often stronger than the non-specific interaction. Predicting the severity of the disease by measuring the ratio of levels and affinity of at least two immunogenic proteins is the core of the invention. Additionally to this core of the invention, a simultaneous but partial elution of the antibodies bound to the specific antigen will reveal easily the difference between the off-rates of antibodies bound to the immunogenic proteins or antibodies bound non-specifically. This not only reveals the strength of binding of the three isotype antibodies (M, G, A) to at least two immunogenic proteins of the virus but also reduces the number of false-positives.

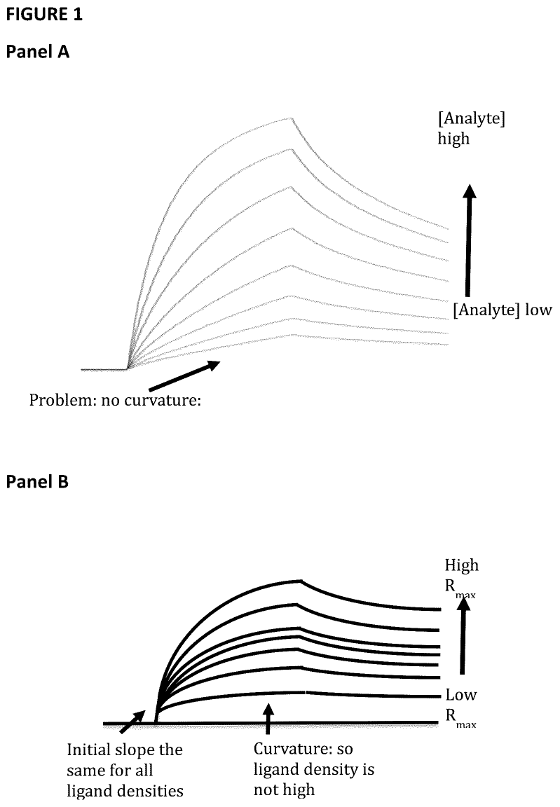

The need for a gradient plug & Play kinetic parameter analysis is shown in FIG. 1. FIG. 1 Panel A represents the current standard procedure with a measurement time of approximately 1 hour including regeneration. An overlay plot of eight analyte injections in serial dilution have the same ligand density for all curves, initial slopes are different. This results in a curvature ligand density that is too high with measurements of the wrong kinetic constants. The present invention provides for measurement times of approximately 5 minutes without regeneration (FIG. 1 Panel B). From a single analyte injection, initial slopes for all curves are the same while ligand density is different. Thus, the ligand density is not high. Assessment requires looking for the location on the gradient where Rₘₐₓ=50RU. By analyzing the kinetic parameters in this way, a single measurement is sufficient.

Accordingly, the present invention provides for a method and device for assessing concentration and avidity detection of antibodies against infectious diseases measured with evanescent field based biosensors using a continuous gradient of ligand densities.

### Surface Plasmon Resonance Imaging and Spotting Instruments

The IBIS MX96 instrument (IBIS Technologies, Enschede, The Netherlands) applies a valve-less consecutive injection of samples. “Back-and-forth” flow-based fluidics enables unlimited interaction times using a sample of 100 microliter. Further, the MX96 applies in the dissociation phase so-called co-pumping to keep the concentration of dissociating analyte zero to reduce rebinding effects. The continuous flow microspotter (CFM, Wasatch Microfluidics, Salt Lake City, Utah, USA) enables high reliability printing of ligand molecules under flow conditions. SensEye® sensors (gel-type E2S, Ssens, Enschede, The Netherlands) using preactivated surface chemistry or streptavidin coated sensors were applied for printing an array of ligand samples. The instrument enables multiplex, up to 96 spots, kinetic analysis of interactions. The method of the invention for RBD and NCP cannot be applied to the MX96 instrument because the sensor applies a hemisphere and optically cannot be replaced to create a double gradient on the same sensor using a single flow cell. If in the MX96 system double flow cells are applied or flow cells that can be moved then it will also work with the MX96. However, in preferred embodiments of the invention the sensor prism rather than the flow cell will be replaced and the flow cell position will be kept in place, which has additional advantages.

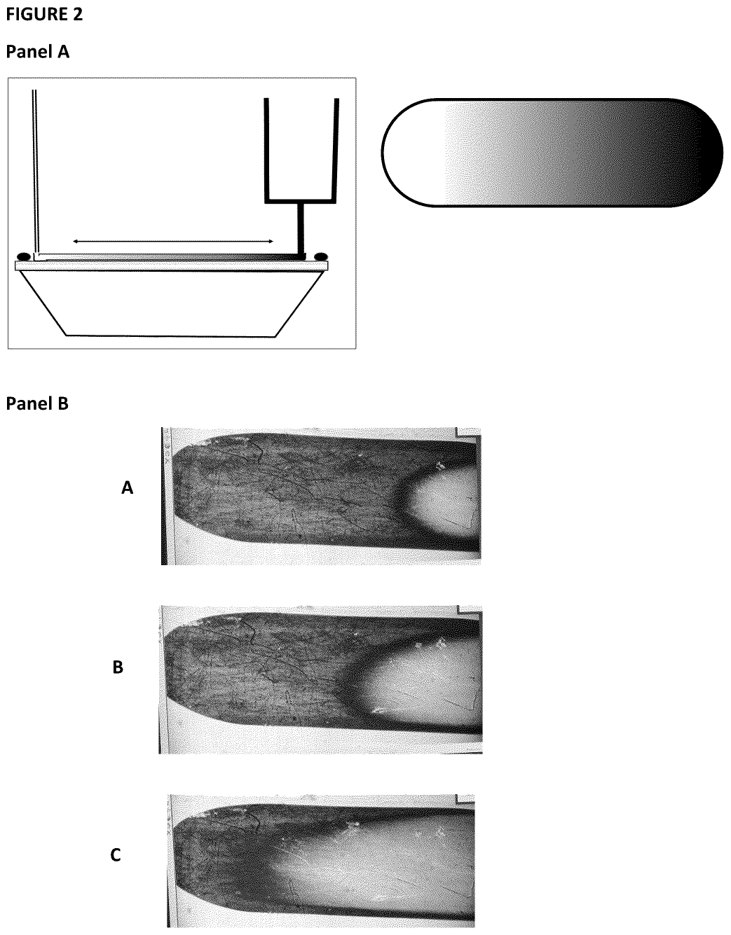

Thus, specific aspects of the several embodiments in the present invention are as follows. As shown in FIG. 2 Panel A, a gradient can be created on a sensor prism using contact time differences where the ligand is immobilized more on the right than on the left. The ligand will flow from one direction and back to enable contact time differences. FIG. 2 Panel B depicts the contact time gradient in the flow cell. An SPR image of injecting a ligand from the inlet on the right of the flow cell is depicted at Panel B (A). The sensor surface is exposed to a ligand solution entering the flow cell. On the right side of (A), the resonance conditions are changing during injection. The injection of ligand solution at a later stage is depicted in Panel B (B). The ligand solution is slowly flowing from right to left. Panel B (C) is similar to (B) but almost at the end of the flow cell. In this way, a gradient of ligand density is created by contact time differences from inlet to outlet. At the inlet, there will be maximum ligand density and at the outlet zero ligand density.

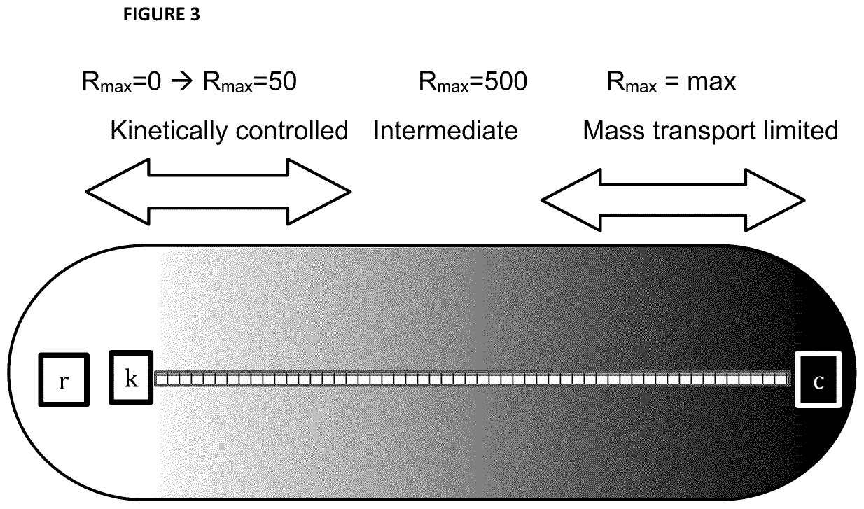

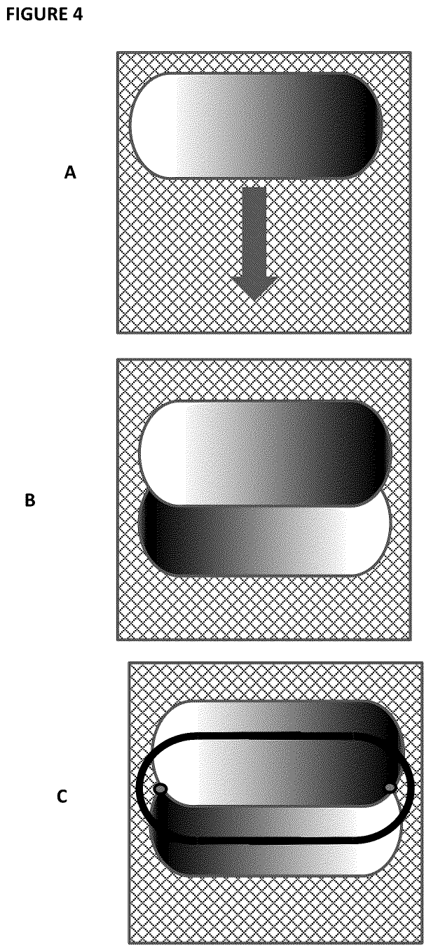

In the gradient, there are three regimes for measuring biomolecular interactions (see FIG. 3). Regime (1) is a kinetically controlled regime for affinity parameters at fixed by low Rₘₐₓ. Regime (2) is an intermediate regime and regime (3) is a mass transport limited (MTL) regime at high ligand densities for concentration measurement using slopes under MTL. Here in the middle of the gradient, abundant measurements will be generated and software will find the right condition at Rₘₐₓ=50 at square “k” for measuring the kinetic parameters. The software will find location “k” on the gradient by analyzing a high number of interactions on the gradient. At square “r” there will be no ligand density and this will be used as reference. At square “c” is the location to measure the concentration at MTL conditions using the initial slope after injecting the analyte in the flow cell. This analyte is exposed to all regions at once. FIG. 4 depicts a representation for the generation of two gradients in opposite directions and a mixture of the two gradients in the middle (A). The first gradient is created at a location out of center of the sensor, ⅓ of the width of the flow cell (B). Exposure ligand 1 with different contact times where the sensor is rotated 180° and the sensor is moved down for ⅔ of the width of the flow cell. Exposure ligand 2 with different constant times. Finally, the flow cell is placed on the three sections by moving the sensor holder to the center position (C).

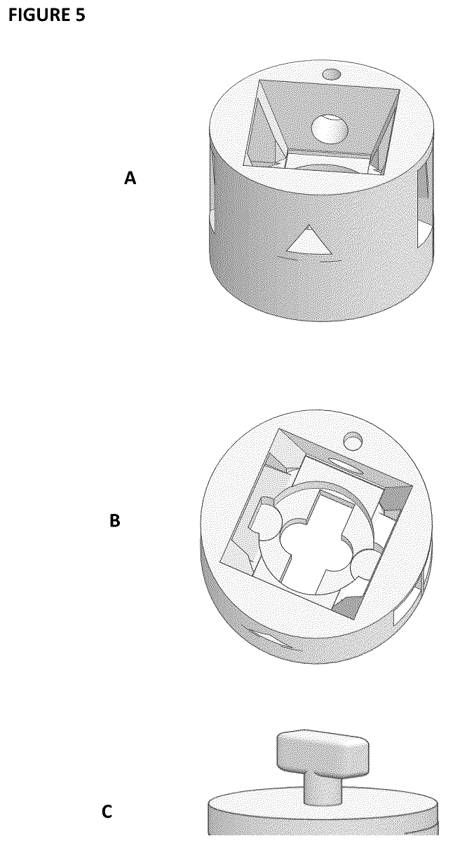

A still further embodiment is showing in FIG. 5. The holder for the sensor prism for printing a double gradient is shown in a top perspective (A) with the positioning cross (B) for replacing the position of the sensor in the optical beam. A bayonet closure (C) is used for placing the sensor in the instrument.

### Multiplex Measurements to SARS-CoV-2 Antigens to Determine Polyclonal Affinity

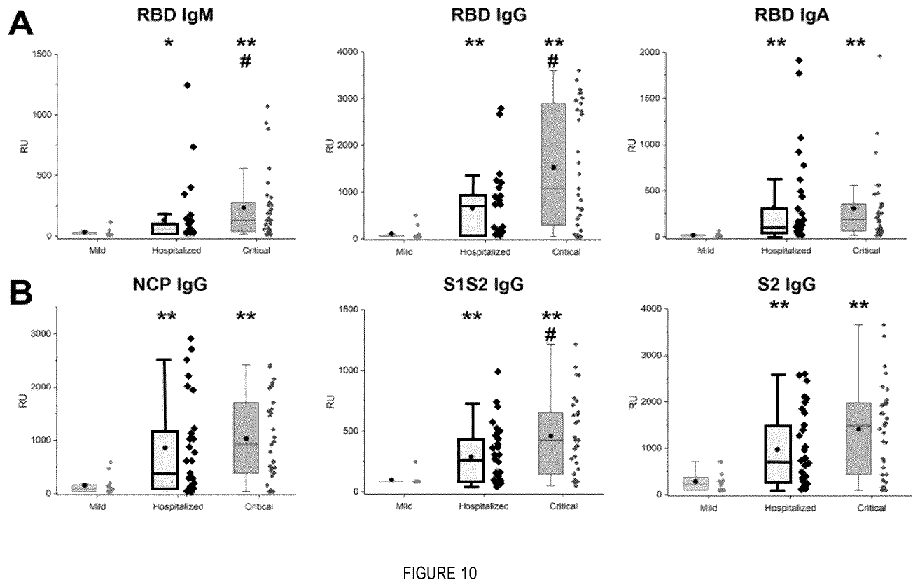

Antibody interactions affinity with SARS-CoV-2 antigens were determined as described previously (FIG. 10). Briefly, patient sera were injected under flow to a multiplex-coated SARS-CoV-2 antigen sensor, with a 3 min association and 1 min dissociation time. The 1:1 Langmuir interaction model is applied to determine affinity constants of the polyclonal antibody pool. The exact value of the equilibrium dissociation constants (apparent K_{D}) cannot be determined because the concentration of the polyclonal antibodies in the serum is unknown and the binding curves do not show monophasic behavior. In our previous investigations, we found a direct correlation of the value of the equilibrium dissociation constant (K_{D}) with respect to the off-rate (k_{d}). In our assay, the exact value of the dissociation constant (k_{d}, s⁻¹) for the overall binding antibodies can be determined after 30 seconds in the dissociation phase because the ligand density (in RU) can be measured accurately by dividing the slope with the response. To measure off-rate solely as an indicator of binding strength is perhaps oversimplified because the ligand density has a large effect. It is better to measure on-rate and off-rate and using a gradient to measure both concentration on the high ligand density location and the kinetic rate values at a low but fixed Rₘₐₓ value (e.g. Rₘₐₓ is 50 RU) and this is the core of the invention as described in this specification.

### Sensor Preparation

For SPRi measurements the multiplex SPR imaging instrument (IBIS MX96, IBIS Technologies, Enschede, the Netherlands) and the Carterra LSA platform (Salt Lake City UT, US) were used with an installed sensor prism (HC30M, Xantec Bioanalytics Düsseldorf, Germany) but to practise the invention any evanescent field based biosensor technique can be applied. Similar results for both instruments were obtained with this sensor surface. The sensor was prepared by first stabilization and removing the protective layer in water, followed by treatment with a 1:1 aqueous solution of 100 mM N-hydroxysuccinimide (NHS) and 400 mM N-ethyl-N′-(3-dimethylaminopropyl) carbodiimide hydrochloride (EDC) for 10 minutes. After rinsing with water for 20 seconds, the sensor was exposed for 20 minutes to the Spike RBD/NCP his-tag (SINO biological Frankfurt, Germany) in immobilization buffer (50 mM sodium acetate pH 4.8) or the his-tagged N-protein (NCP) (SINO biological Frankfurt, Germany). Coupling with EDC-NHS yielded a reproducible sensor surface. After rinsing the sensor for 20 seconds with water the surface was passivated with 1M ethanolamine (pH 8.5) for 10 minutes. The sensor was then equilibrated in the running buffer composed of PBS (137 mM NaCl, 10 mM phosphate, 2.7 mM KCl, pH 7.4) supplemented with 1% bovine serum albumin (BSA) and 0.5% Casein and 0.1% Tween 20.

### Spotting Sera

Dilution of the serum samples was constant in an optimized dilution ratio of 1:100. Each 2 μl serum was diluted with 198 μl of running buffer and pipetted in a 96 wells plate. For measurements on the IBIS MX96 a Continuous Flow Microfluidic (CFM) system (Carterra Salt Lake City UT, US) was used to capture 96 sera on a sensor functionalized with RBD/NCP-Spike coupled. The upper part of the flow cell can be addressed with RBD and the lower part with NCP or vice versa. The first 48 samples were spotted in duplicate for 3 minutes on the top (RBD) section and subsequently to the down (NCP) section. The Carterra LSA enables printing of 384 spots as 4 nested positions of 96 each. Here the sensor can be immobilized with RBD on top and NCP on the bottom section of the sensing area. Equal sera are supplied to the top section and to the bottom section. While the operation and injection of sample is similar for both instruments, the spotting process and dissociation rate can be followed in real time on the LSA. However, for both the IBIS MX96 and Carterra LSA only the dissociation rate can be determined and not the full affinity data including on-rate and equilibrium dissociation constant. One will get deviations in the off-rate when the ligand density is not optimally tuned. This is now circumvented when a gradient of ligand density is applied as described in this specification.

### Measurements on the IBIS MX96

The SUIT (Set Up Ibis Tool), DAX (Data acquisition software) and SPRINTX (Analysis software) software packages on the SPRI MX96 were used. After washing of the sensor chip spotted with patient sera in two concatenated sessions for the top (RBD) and down (NCP) section, the sensor was first incubated with 50× diluted goat-anti-human-IgM (algM approximately 4 mg/ml, 20-S5170 GND1-D0 Fitzgerald) in running buffer (200 μl for one run) and the second a 100× diluted goat-anti-human-IgG (algG-Fc approximately 8 mg/ml, 20-S1211G001-S4 Fitzgerald) in SPRi running buffer. The third injection was with a 100× diluted goat-anti-human-IgA (algA approximately 7 mg/ml, 20-S1111G000-S4 Fitzgerald). After converting the data by local referencing, zeroing the baseline and aligning the injection points of the three injections, the Rₘₐₓ value was determined using a special biphasic fit algorithm (InterFluidics, Haaksbergen, The Netherlands). This software tool programmed using Microsoft ‘R’ Studio allows calculating the data on both SPR imagers. If the curve did not show an exponential behavior (e.g. negative samples) then a linear fit was applied and the average value of the linear fit was determined.

### Off-Rate Measurements on the LSA

In total 48 selected serum samples were spotted in duplicate in a single run on the HC30M RBD coupled and NCP coupled sensor prism surface in 4 dilutions (1:50, 1:100, 1:200 and 1:400) to generate a 384-array. During the spotting process, the binding signals are followed for 15 minutes and each serum sample was measured 8 times at 4 dilutions. The signal recorded in RU mirrors the total anti-RBD/NCP antibodies bound. Following the spotting process, a 5 min injection of RBD-NCP (15 μg/ml) in dilution buffer resulted in sufficient dissociation of the anti-RBD antibodies and anti-NCP antibodies. For all 384 spots, the global dissociation- or global off-rate constant can be calculated. The final step consists of sequential injections of solutions of anti-IgM, anti-IgG and anti-IgA antibodies. The ratio of bound immunoglobulins regarding RBD and NCP can be calculated by determining the Rₘₐₓ values from the anti-isotype antibodies binding signals. The Rₘₐₓ value has a direct relation with the captured ligand density and so with the concentration of anti-RBD and anti-NCP antibodies in serum.

Effective methods for predicting COVID-19 disease trajectories are urgently needed. Additionally, to our RBD/NCP ratio observation, very recent evidence has been found that non-neutralizing antibodies generated against a 21-residue epitope from nucleocapsid (termed Ep9) are associated with severe disease, including admission to the intensive care unit (ICU), requirement for ventilators, or even death. Importantly, anti-Ep9 antibodies can be detected within six days post-symptom onset and sometimes within one day using an enzyme-linked immunosorbent assay (ELISA) which has similar sensitivity than SPRi. Furthermore, anti-Ep9 antibodies correlate with various comorbidities and hallmarks of immune hyperactivity and sepsis. So, COVID-19 patients with high titers of low affinity antibodies are associated with disease severity.

### Results

### Simultaneous Measurement of 48 Samples for Anti-SARS-CoV-2 IgM, IgG and IgA Antibodies by SPRi Against RBD and NCP

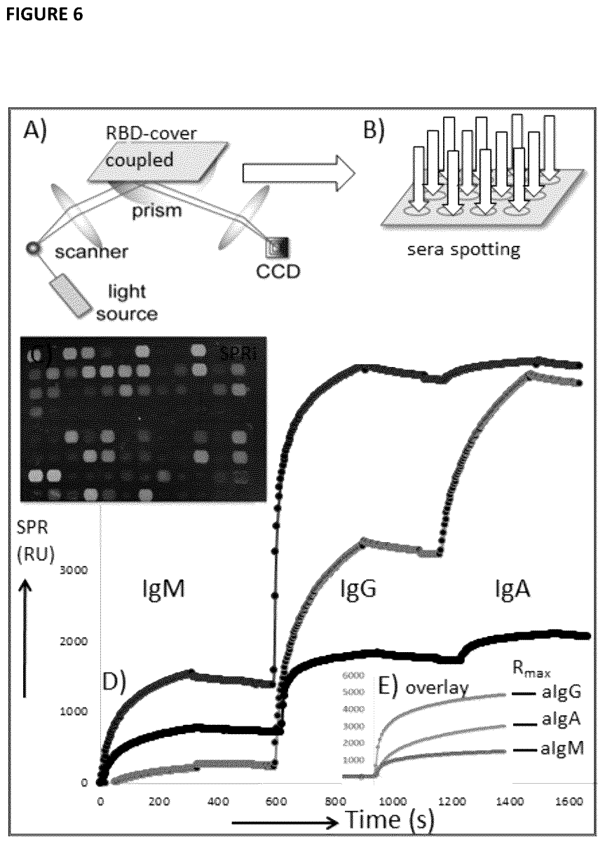

FIG. 6 shows the principle of the SPRi assay for determining the isotype fractions. In Panel A, 96 sera are spotted on an RBD/NCP coupled sensor. In Panel B, the sensor is placed in the SPR imager and real-time measurements were performed during three concatenated injections of anti-IgM, anti-IgG and anti-IgA. Panel C shows the SPR reflection image after the injection of the anti-isotype antibodies of the 96 sera. Panel D shows typical sensorgrams of 3 sera. The first curve represents a serum with high IgM (Rₘₐₓ1940 RU), very high IgG (5012 RU) and weak IgA (243 RU). The third curve shows a serum with moderate IgM (845 RU), a moderate IgG (1215 RU) and a weak IgA (464 RU). The second curve shows a serum with a weak IgM (203 RU) a high IgG (3950 RU) and a high IgA (3796 RU). Panel E shows a patient overlay for calculating the Rₘₐₓ values of anti-IgM, anti-IgG and anti-IgA which is proportional to the bound IgM, IgG and IgA anti-RBD/NCP. The baselines are zeroed and the injections are aligned. This enables the application of a biphasic binding model for calculation of Rₘₐₓ for the three isotypes.

### Immune Response Characterization of COVID-19 Patients Using SPRi

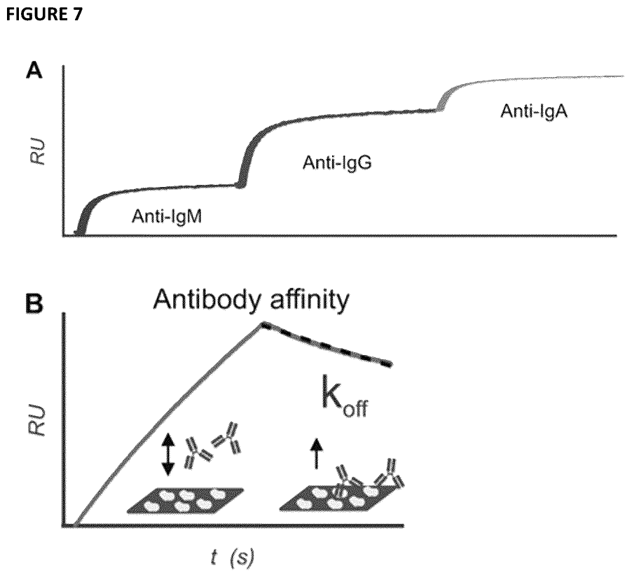

To study the immune response of COVID-19 patients, SPRi technology was used as described previously. In brief, the IgM, IgG and IgA binding was real time determined against specific SARS-CoV-2 proteins (RBD/NCP, NCP, S1S2 and S2) (FIG. 7A) and the affinity of the polyclonal antibody pool to each protein was measured to assess strength of binding (FIG. 7B). Moreover, binding of patient antibodies to specific epitopes on the RBD/NCP protein using a peptide library was investigated (FIG. 7C).

Sensorgrams were measured for all samples simultaneously and shown as an overlay plot in FIG. 7. Repetitive measurements using the same sera showed that the RU level variation was less than 5%.

### Strength of Binding Measurement of Anti-SARS-CoV-2 Spike RBD/NCP IgG, IgA and IgM

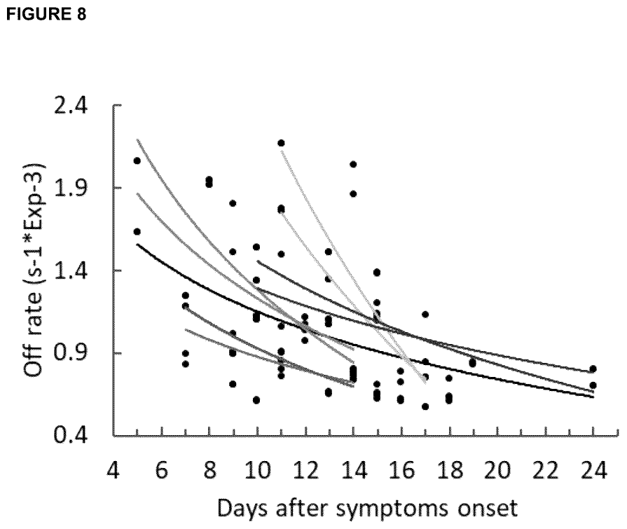

Ligand density can cause analyte to rebind during its dissociation. Rebinding results in an overestimation of the off-rate value or stronger binding. To reduce the rebinding effect of dissociating molecules, we added free RBD/NCP in a concentration of 15 μg/ml to the running buffer. In 5 minutes, we observed a mixed degree of dissociation of the various and longitudinal samples (see supplementary FIG. 7) and the dissociation or off-rate constant can be calculated and plotted as a function of the days of symptoms onset (see FIG. 8). During the development of the disease, we observed a smaller off-rate indicating that the avidity or quality of the antibodies improves. So, the patients are producing a better-quality repertoire of polyclonal anti-RBD/NCP antibodies over time. For all longitudinal samples this trend in off-rate is observed (strength of binding becomes better). A discrepancy exists that only the weakest bound antibodies are dissociating first from the sensor surface. Weak binding antibodies will block the RBD but the neutralizing effect is decreased. When a gradient of ligand density was applied in this type of measurement then the addition of free RBD/NCP was not necessary because the software will always find the right condition at Rₘₐₓ=50 for measuring the kinetic parameters on-rate and off-rate. The on-rate needs the concentration parameter and this is measured under mass transport limited conditions at high ligand density on the same gradient.

SARS-CoV-2 binds the ACE2 receptor more strongly in comparison to SARS-CoV. This implies that antibodies need to have high affinity to compete and neutralize the virus. Our method for profiling the immunity in terms of isotype concentration and strength of binding enables to reveal this effect in a high-throughput manner. High avidity anti-RBD antibodies at low concentration are perhaps more effective for neutralizing the SARS-CoV-2 than a higher concentration anti-RBD antibodies with lower affinity. Anti-NCP antibodies will not neutralize the virus and can be used as indicator of infection with respect to vaccinated people who develop only anti-RBD antibodies.

From the previous experiments, it is clear that the binding of antibodies from different patients show a great diversity. This parameter is, to our knowledge, never applied in a clinical testing for specific antibody monitoring and for predicting COVID-19 severity.

The results are the basis for a new type of COVID-19 prognostic biomarker in addition to the full NCP protein and S-RBD using label-free sensing technologies like SPR to allow early identification and triage of high-risk patients. Such information including the avidity will lead to effective therapeutic intervention. A patient with mild symptoms shows low titers, but the anti-RBD and anti-NCP can easily be detected. During our initial study, we could not measure vaccinated people, but many papers show that anti-NCP antibodies are not present in the sera of vaccinated people without SARS-CoV-2 infection. Although evidence with respect to the ratio of anti-RBD and anti-NCP is shown in our latest paper for correlation with severity of the disease, it is clear that vaccinated versus non-vaccinated people (seropositive and seronegative) can clearly be identified. Additionally, the anti-RBD plus anti-NCP value correlates well with the severity of the disease regarding seropositive patients, and the anti-RBD value of vaccinated people (so negative for NCP) shows the status of immunity for COVID-19. Epidemiologic studies showing correlation with respect to vaccinated and seropositive patients are not necessary but only validation of patient data with traditional serology tests (e.g., ELISA).

### Total Immune Response of SARS-CoV-2 Antigens Per Patient Subgroup.

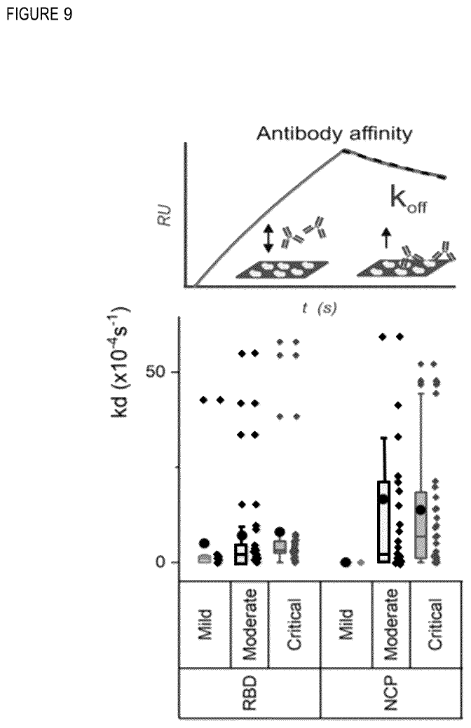

The multiplex SPRi measurement of four SARS-CoV-2 antigens was used to determine the total immune response (IgM, IgG, IgA) per patient. Analytical sensitivity was sufficient for the IgA, IgM and IgG response to RBD, and the IgG response to NCP, S1S2 and S2 protein while IgA and IgM responses to these antigens were not. FIG. 9 shows the immune responses stratified by mild, moderate or severe/critical disease severity. Patients with moderate or critical disease showed significantly higher responses of IgM, IgG and IgA against RBD and IgG against NCP, S1S2 and S2 than patients with mild disease. In addition, patients with critical disease had significantly higher RBD-IgM, RBD-IgG and S1S2-IgG compared to patients with moderate disease. Thus, increased disease severity was positively associated with an increased titer of IgG antibodies to the respective antigen also with increased titers of IgM and IgA antibodies.

### Binding Strength of the Antibodies.

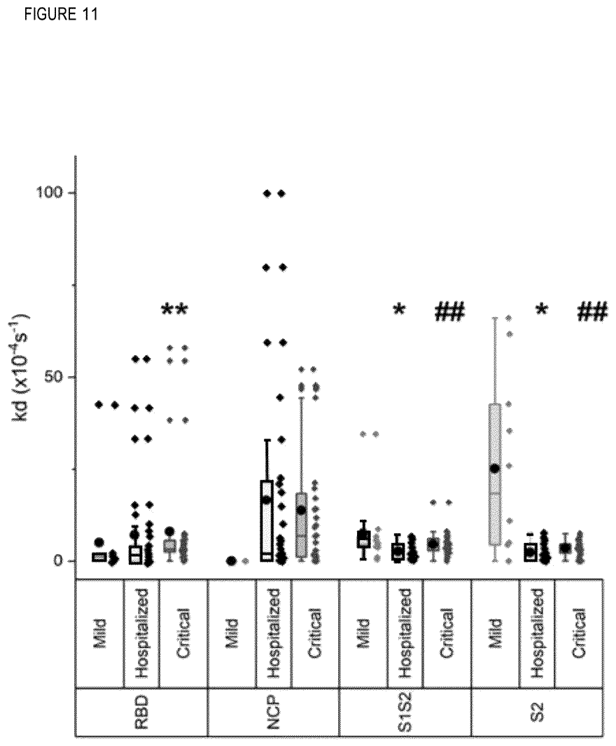

The off rate (k_{d}) was determined to rank the binding strength of the polyclonal antibody pools reacting with the respective antigen. A higher k_{d} correlates with a higher equilibrium dissociation constant (K_{D}) and therefore a lower affinity. FIG. 8 shows increasing k_{d} with increasing disease severity, which was significant for critically ill patients vs mild for RBD and NCP, and critical vs moderate as well as mild for S1S2 and S2, suggesting decreased maturation towards antibodies with higher affinity. An exception to this is the lower measured antibody affinity toward S2, and in lesser extent towards S1S2, in mild compared to moderate and critical patients (see FIG. 11).

### Immune Response, Gender and Binding Strength.

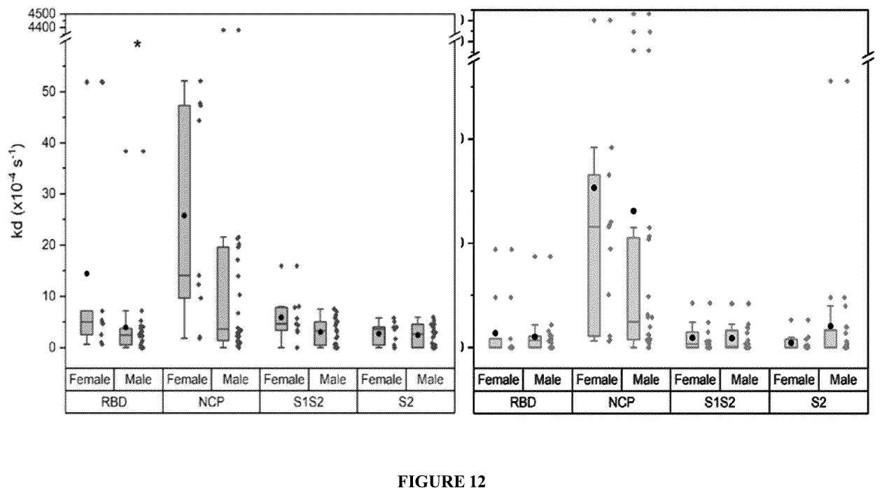

Already since the beginning of the COVID-19 outbreak it was observed that men were more at risk for worse outcomes and death, independent of age. Discriminating the immune responses of men (n=42) versus women (n=28) no differences were found regarding amounts of antibodies (FIG. 12 S1), specificity of antibodies and binding strength of antibodies. (data not shown). Only in the group with moderate disease the anti-RBD/NCP antibody pool in men had significantly lower binding strength than in women (FIG. 12 S2).

In a later study, we compared the data between critical and moderate patients and observed that the strength of binding towards all antigens is lower with increasing disease severity.

Thus, although in critically ill patients more antibodies to RBD, Spike and NCP are produced than in moderate patients the binding strength is much less. As the affinity of the RBD domain for ACE2 receptor is very high (approximately 10 nM), this lower affinity is likely to have severe consequences for effective neutralization. Additionally, to the correlation of the severity of COVID-19 using antibodies levels and strength of binding, also strong correlation exists with the expression of high levels of D-dimer, C-reactive protein (CRP), Interleukin6, among others. These biomarkers can be implemented in the multiplex SPRi test for further in-depth analysis of COVID-19.

### The Invention for Determining COVID-19 Severity

The assay of the invention can be performed very simply. Already from at least two immunogenic proteins that are addressed in the flow channel in a gradient of any multichannel biosensing imaging instrument the COVID19 serological sample in a dilution of 1:100 can be exposed to the at least two proteins. However, the more specific immunogenic epitopes the better the immune profile regarding severe COVID-19 can be assessed. The binding of the specific antibodies of the serum can be followed in real-time in 3 minutes and the dissociation can be followed in real-time for two minutes to any of these specific epitopes. The ratios of initial slope as function of the concentration can be calculated and the value of the slopes of the dissociation phase can be calculated for the antibodies against the at least two immunogenic proteins.

It is also worthy of mention that this approach can be readily applied to monitoring immune response in other types of infectious disease as well. Any protein targeted by an immune response can be immobilized to the sensor surface in a gradient allowing for a quantitative, and reproducible means of characterizing immunity and applying at least two immunogenic proteins. The more the better for in depth profiling but as such we found that the RBD vs NCP levels and strength of binding is already sufficient to predict COVID-19 severity. The real-time monitoring of signals in SPRi makes it well suited too for rapid deployment and optimization. In our latest findings, not only the level of concentration is important only for prediction of COVID-19 severity but also the ratio between avidities of the specific antibodies to the immunogenic proteins.

In this workflow, the effect of the affinity/avidity of antibodies can be ranked and quantified precisely with the goal of improving clinical outcomes. In addition to following the strength of binding and concentration of anti-RBD/NCP antibodies for COVID-19 patients, this assay is ideally suited for monitoring of healthy people who are vaccinated against SARS-CoV-2. Only anti-RBD antibodies are measured while anti-NCP antibodies are not present. The SPRi assay described here can provide critical insights in determining if the final quality of the IgG response after vaccination is adequate to generate neutralizing antibodies with sufficient affinity for clearing the virus.

Additionally, in order to gain the highest success rate in developing therapeutic neutralizing mAb's, individuals and donors for passive immunization programs should be screened for the highest avidity immune response against the immunogenic proteins of SARS-CoV-2.

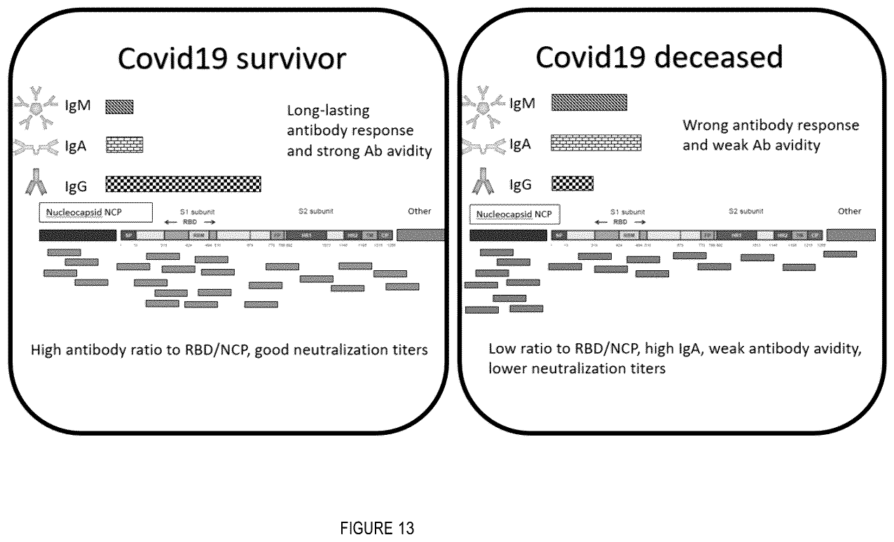

The method to measure the avidity constants of at least two immunogenic proteins as described in the present specification is reliable, independent of concentration, high throughput and accurate for profiling the immunity of patients. It predicts COVID-19 severity. Additionally, it reduces the number of false positives dramatically. The method revealed the trends of maturation and enables the assessment of the overall quality of the antibodies (see FIG. 13).

### Advantages of Evanescent Field Based Optical Biosensor Technologies for COVID-19 Severity Monitoring

Other evanescent field based optical biosensor technologies as Surface Plasmon Resonance imaging, among others or any other label free, real-time optical biosensing imaging technology (grating couplers, holographic imaging, Attenuated Total Reflection imaging etc.) can be applied to follow the interaction of specific antibodies in real-time within three minutes. This results in the fastest fully quantitative multiplex assay with respect to any other labeled technology (e.g. Elisa, EIA, ECLIA, Lateral Flow Assays etc.). We found a strong correlation of anti-Nucleocapsid antibodies with respect to anti-RBD and strength of binding with COVID-19 severity. Only label-free sensing technologies are able to measure this parameter in a 5 minutes time window, 3 minutes association and 2 minutes dissociation). SPR imaging in multiplex mode enables full profiling of a patient sample.

### Discussion

COVID-19 patients demonstrate large heterogeneity in disease severity as result of SARS-CoV-2 infection. Literature suggests the humoral immune response is implicated in the disease severity, yet the relationship is not fully understood. We have developed a number of assays based on SPRi to more broadly characterize patients' humoral response and increase our understanding of its contribution to disease progression. We have previously shown SPRi can be used to detect the composition and affinity of the antibody response to SARS-CoV-2 in patients.

In this study, serum samples from 76 SARS-CoV-2 patients were analyzed with SPRi using the viral antigens NCP, S1S2, S1 and RBD. For all antigens, significantly higher amounts of antibodies were found in the patients with moderate or critical disease severity versus the patients with mild disease. The data further shows significant differences between the categories critical, moderate and mild for IgM, IgG and IgA isotypes. Our results confirm similar findings as reported in other serological studies on SARS-CoV-2 patient cohorts. In addition to measuring the immune response, SPRi enables us to measure the strength of antibody binding. Recently, we demonstrated that the off rate (kd) correlated well with the affinity equilibrium constant (KD) and as such can be used to rank the antibody response in terms of binding strength. Remarkably, patients with critical disease had significantly lower strength of binding antibodies to the RBD in comparison to mild patients and low strength antibodies to NCP were seen in the moderate and critical patients only. This contrasted with binding strength data towards S1S2 and S2, which show higher binding strength towards moderate and critical vs mild. An important consideration here however is that the mild patients have rather small antibody responses, making affinity measurements less precise. When we compared the data between critical and moderate patients only, we see that affinity towards all antigens is lower with increasing disease severity. Thus, although in critically ill patients more antibodies to RBD, S1S2, S2 and NCP are produced than in moderate patients the binding strength is much less. As the affinity of the RBD domain for ACE2 receptor is very high approximately 10 nM), this lower affinity is likely to have severe consequences for the effective neutralization.

Most neutralizing antibodies isolated from SARS-CoV-2 convalescent donors target the RBD, and a subset of these antibodies blocks viral entry by binding to the ACE2-binding site of the RBD. Antibodies to RBD displaying low affinity may not be able to compete with the interaction between RBD and ACE-2 resulting in more severe disease. Beyond neutralization, antibodies can elicit an array of Fc-mediated immune function such as antibody dependent complement deposition, cellular phagocytosis and cell-mediated cytotoxicity. Although these are considered to be beneficial functions, they can also induce inflammation during viral infection. It has been described that severe COVID-19 patients had higher antibody levels and more antibody dependent complement deposition but less antibody dependent cellular phagocytosis and cytotoxicity. This indicates that not only quantities of specific antibodies matter but that qualitative properties play an important role as well. Furthermore, we found binding of IgA only in critically ill patients. This supports the finding that prolonged IgA response is associated with unfavorable clinical outcomes.

Already since the beginning of the pandemic it was observed that men are more at risk for severe disease, worse outcome and death. However, in our cohort the antibody response of men and women was equal. We did not find differences in amount of IgM, IgG or IgG between men and women (data not shown). Similarly, the binding strength of the antibodies in men and women with critical disease did not differ. Only in the group with moderate disease, the binding strength of anti-RBD/NCP antibodies in male was lower than in women. This indicates that the primary antibody response between male and female is not solely responsible for the difference in disease severity. It is possible that downstream sex differences in innate immune system responsiveness, for instance the complement system, might play a critical role.

The SPRi assays allowed us to characterize the details of the humoral response, including isotype, affinity and epitopes on a single platform. We applied this to characterize COVID-19 patients, however this could be applied to any infectious disease. We showed a strong correlation between antibody concentration and disease severity. Yet, these antibodies are of reduced affinity, and maturation on neutralization epitopes might be dysfunctional.

Given the increase of k_{d} with increasing disease severity of specific antibodies and incomplete isotype switching, we hypothesize that in a subset of patients, antibody production and maturation is ineffective, resulting in a polyclonal antibody pool of lower affinity. As a result, neutralization of SARS-CoV-2 is less effective leading to a higher viral load and increased inflammation. Moreover, in an attempt to effectively neutralize the higher viral count, antibody production increases and serum levels rise. In neutralization assays, higher antibody titers may also compensate for lower affinity but this might come at the expense of antibody induced side effects, since these antibodies will find abundant targets potentially leading to Fc-mediated immune responses, including activation of complement cascades, increased coagulation and activation of innate immune cells. Together, this contributes to a hyper-inflammatory state and might be implicated in the disease severity of COVID-19 patients.

### The SPR Imaging System to Determine Kinetic Parameters Using a Ligand Density Gradient.

By supplying the ligand solution under controlled conditions it enables also to generate a steep gradient of ligand density on the sensor surface. This has a huge advantage for measuring affinity parameters, because the value of the affinity constants (k_{d}, kₐ, and K_{D}) that are determined by label free interaction analysis methods are affected by the ligand density [6]. By creating a gradient in ligand densities an SPR imager using the controlled injection flow method it can measure the analyte ligand binding in a spatially resolved manner on the gradient of ligand density. A kinetic titration experiment without a regeneration step can be applied for various coupled antibodies in a gradient ligand density binding to a single antigen. Globally fitted rate (k_{d} and kₐ) and dissociation equilibrium (K_{D}) constants for various ligand densities and analyte concentrations can be measured and parameters can be determined at a fixed ligand density (better a fixed Rₘₐₓ value) e.g. at Rₘₐₓ=100 RU response level (K_{D}^{R100}) or at Rₘₐₓ=50 RU response level (K_{D}^{R50}).

These molecular binding constants that are derived from current, immobilized ligand based assays are affected by the immobilized state of the ligand. This causes the thus determined, apparent constants to deviate from the true, “solution” constants due to interfering effects that result from the immobilization of the ligand. These interfering effects include rebinding effects, mass transport limitation, non-specific binding and deviation from the 1:1 model binding. The higher the ligand density, the more pronounced these interfering effects become and it is generally accepted that the ligand density should be applied just above the limit of detection of the biosensor instrument. The same holds for the analyte concentration—interfering effects will occur when multiple analyte molecules compete for interaction with a single immobilized ligand molecule.

So, the calculation of the “true” affinity equilibrium constant will become more reliable at lower densities, preferably at a “density” of only a single immobilized ligand molecule acting as a free ligand. Then the contribution of the interfering effects will be zero and will no longer influence the rate- and affinity equilibrium constants. Practically, this condition cannot be measured and by decreasing the ligand density the more noisier and less reliable the sensorgrams become. Additionally, the quality of fits to noisy curves cannot be judged adequately. It should be noted that immobilization artefacts and heterogeneity of surface binding sites should be prevented, for instance by oriented capturing of the ligands by applying high affinity anti-ligand antibodies or using tag—anti-tag interactions.

A so-called K_{D}^{R0} method for the determination of affinity constants has been published [7] in which the contribution of interfering effects is minimized or theoretically zeroed, so that the constants are a better estimate of the true constants of bio-molecular interactions in solution. This method is based on the extrapolation of the number of immobilized ligand and analyte molecules to zero, thus mimicking the interaction in which only one ligand and one analyte molecule are involved, enabling a true 1:1 binding model with theoretically not any interfering effect.

Recognized practical effects are additional ligand immobilization artefacts and heterogeneity of surface binding sites. The method will not compensate for this and the alternative route is by capturing ligands followed by the target interaction. When a harsh regeneration step is included the Rₘₐₓ value will decrease after the subsequent injections of the analyte concentrations and can again affect the kinetic affinity constants. Preferably any regeneration step of the surface should be avoided and this is achieved using kinetic titration.

Calculation of the kinetic constants was from spots with discrete ligand densities. Nowadays many users of SPR platforms are tuning the ligand density in such a way that the interaction with the analyte is at very low but still measurable values. The sensitivity of the instrument determines how low the ligand density can be. A user determines what he thinks is a low value and the values that users are creating are Deviating from each other because there is no rule to interpret the quality of fitting of the binding curves.

According to the invention a steep gradient of ligand density is created and the instrument measures the analyte binding on the ligand gradient. All densities are available from very high to zero low. So if the gradient in the flow cell is divided in e.g. 1000 Region of Interest or better a tunable or dynamic Region of interest then the instrument can automatically find the binding result at e.g. Rₘₐₓ=100 RU or 50 RU or any value for a similar set of biomolecular interactions. The proven method as published [6] can now be performed on a gradient ligand density instead of on a discrete low ligand density but on a limited number of spots. The interpretation of fitting quality by a user e.g. by applying a 1:1 Langmuir binding algorithm is not necessary anymore. The software generates the biomolecular affinity parameters measured always in the same way using the same ligand density at a location somewhere on the gradient. Interpretation of curves by a user, lab technician or operator of the instrument is not necessary anymore. Always the parameters are generated in the same way with the dynamic gradient method which is a huge improvement in analysis of the data.

There are many more applications when a controlled gradient of ligand density can be created on the sensor surface. E.g. particles like cells, viruses, organelles, vesicles etc. contain a certain number of cell surface receptors (CD's) that bind to anti-CD antibodies. These antibodies will bind these particles and tests like an inhibition test can be performed on the gradient. The higher the affinity (or, better, the avidity for multivalent interactions) of binding the better these particles will be present at low ligand densities. At ligand density zero it will not bind.

The T/S measurement strategy published in the Handbook of Surface Plasmon Resonance 2^{nd} edition chapter 12.8.1. page 447 can now be applied on a gradient. This could be an important strategy for avidity ranking of the interactions using the increased flow protocol as described in chapter 12.8.4. page 463. These detection strategies could be better applied to the sensor surface with a gradient of the ligand density.

Cells will bind to the sensor surface after injecting cells in a flow cell. Companies who are developing antibodies for various cell-applications need to characterize the affinity of monoclonal antibodies against living cell receptors. Direct detection of the antibody that binds to a sedimented cell line was not possible because of highly unstable baselines due to activity of the cells. However we found that the release of cells from the sensor surface depends on several factors. E.g. the flow velocity, the number of receptors on the cell, the affinity of the cell receptor to immobilized ligand, the ligand density etc. are important parameters. When a ligand gradient is applied in combination with increasing flow rates (shear rate) then ranking the affinity could possibly be measured on multiple receptor—Ab combinations. The shear on cells depends on the local velocity profile of the buffer stream on the immobilized cells. At a certain area on the ligand gradient the cells will still bind but by increasing the buffer velocity that drag the cells from the surface the cells will not bind anymore. The higher the velocity the higher the ligand density that is needed to keep the cells on the surface. With SPR-imaging this process can be followed in real time. By addressing a uniform force on the cells, a ligand density series of anti-membrane antigens will tune the position where cells at a certain velocity will dissociate from the gradient. In this way affinities of receptors on cells can be compared and ranked to each other when simultaneously different antibodies are immobilized in a ligand gradient. Then this SPRi-application will gain enormous impact.

A reliable and multi-functional SPR imaging measuring method is obtained when preferably the sensor surface comprises a plurality of active sites (preferably in a continuous gradient) monitored for change in the surface plasmon resonance angle of light incidence at the sensor surface, preferably with a camera.

The SPR measurement may be carried out in one single flow cell or in a plurality of flow cells e.g. 2 to 6 or more. When a plurality of flow cells is used, then each flow cell may be served by its own pump means for creating the ligand density in a gradient on the sensor surface. However, it is preferred that the flow cell to inject the analyte is served by common pump means such that all spots are subjected to the same conditions (flow rate and transport and passage of sample, buffer therefore making it possible to do a reliable automatic measurement on ligand gradient). This is the so-called “one over all” method.

Mentioned and other features of the SPR measuring system and of the method for SPR measurement according to the invention will be further illustrated by various embodiments which are given for information purposes only and are not intended to limit the invention to any extent, while making reference to the annexed drawings.

The method is important for e.g. point of care measurements for COVID-19 with plug and play features where avidity parameters can be found within 10 minutes without the problems of the ligand density effects. The concentration of the analytes is found at high ligand density where mass transport controlled conditions are occurring. Always in one experiment both concentrations (high ligand density) and avidity (at ligand density where Rₘₐₓ=low e.g 50 RU) can be automatically analyzed. The method could be a breakthrough in point of care detection where both concentration and avidity are important parameters e.g. for infectious diseases in particular COVID-19.

Although the present invention has been described with reference to specific embodiments, workers skilled in the art will recognize that many variations may be made therefrom. For examples in the particular experimental conditions herein described, and it is to be understood and appreciated that the disclosures in accordance with the invention show only some preferred embodiments and objects and advantages of the invention without departing from the broader scope and spirit of the invention. It is to be understood and appreciated that these discoveries in accordance with this invention are only those which are illustrated of the many additional potential applications that may be envisioned by one of ordinary skill in the art, and thus are not in any way intended to be limiting of the invention. Accordingly, other objects and advantages of the invention will be apparent to those skilled in the art from the detailed description together with the claims.

### REFERENCE

1. Jin Y. et al. Diagnostic value and dynamic variance of serum antibody in coronavirus disease 2019. International Journal of Infectious Diseases doi.org/10.1016/j.ijid.2020.03.065 (2020)
2. Brouwer P. J. M. et al., Potent neutralizing antibodies from CoViD-19 patients define multiple targets of vulnerability. Science 10.1126/science.abc5902 (2020).
3. Schasfoort RBM, van Weperen J, van Amsterdam M, Parisot J, Hendriks J, Koerselman M, Karperien M, et al. Presence and strength of binding of IgM, IgG and IgA antibodies against SARS-CoV-2 during CoViD-19 infection. Biosensors and Bioelectronics 2021 2021/07/01/; 183:113165 as doi: https:doi.org/10.1016/j.bios.2021.113165.
4. Schasfoort RBM, van Weperen J, van Amsterdam M, Parisot J, Hendriks J, Koerselman M, Karperien M, et al. High throughput surface plasmon resonance imaging method for clinical detection of presence and strength of binding of IgM, IgG and IgA antibodies against SARS-CoV-2 during CoViD-19 infection. MethodsX 2021 2021/01/01/; 8:101432 as doi: https://doi.org/10.1016/j.mex.2021.101432.Hendriks, Jan, et al. “High titers of low affinity antibodies in Covid-19 patients are associated with disease severity.” Frontiers in immunology (2022): 1620.
5. Schasfoort R. B. M., Editor of the Handbook of Surface Plasmon Resonance, 2nd edition, Royal Society of Chemistry London, UK (2017)
6. Schasfoort R. B. M. et al. Interpolation method for accurate affinity ranking of arrayed ligand-analyte interactions Analytical Biochemistry 500, Pages 21-23 https://doi.org/10.1016/j.ab.2016.01.023 (2016)
7. Schasfoort, Richard B M, et al. “Method for estimating the single molecular affinity.” Analytical biochemistry 421.2 (2012): 794-796.

1. Method for predicting Immune system related disease severity by the combination of specific antibody concentration and strength of binding of all antibodies isotypes from a body fluid sample from a patient comprising:
a. exposing a sample of a patient's body fluid having an Immune system related disease to an immunogenic antigen immobilized in a gradient on a label-free and real-time imaging biosensor;
b. determining a concentration at high ligand density using an initial slope of a binding curve under mass transport limited conditions; and
c. determining avidity parameters at low but fixed Rₘₐₓ conditions on a ligand density gradient,
wherein the ratio of levels and affinity of at least two immunogenic proteins predicts the severity of the infectious disease in the patient.

2. Method according to claim 1 having the label-free and real-time biosensor based on any evanescent field optical phenomenon.

3. Method according to claim 2 where the evanescent field based biosensor is an optical device based on Surface Plasmon Resonance (SPR) imaging.

4. Method according to claim 1, where a controlled injection of a ligand creates a gradient in ligand density by differences in contact time using at least a single back and forth flow of the sample in the flow channel in contact with a sensor surface.

5. Method according to claim 1, where fluidics are designed such that the sample can simultaneously address at least two channels or more in gradients for measuring the concentration and the association rate and dissociation rate simultaneously for at least a duplex measurement of the same sample.

6. Method according to claim 1, where the immune system related disease is an infectious disease.

7. Method according to claim 1 where the immobilized immunogenic antigens are at least a Receptor Binding Domain and Nucleocapsid (NCP) of SARS-CoV-2.

8. Method according to claim 7 where binding constants of the Nucleocapsid antibodies and the binding constants of RBD predict COVID-19 severity of infectious disease in the patient.

9. Method according to claim 1 where half a flow cell is applied for timely exposure of the ligand from inlet to outlet to create a gradient in ligand density of the first immunogenic protein by rotating the sensor 180 degrees to allow a next immobilization with the second protein in a timely exposure from inlet to outlet of the flow cell at the top section while the first gradient is still on the down section whereby the sensor is installed with the multiplex flow cell for measuring the strength of binding and isotypes of the bound antibodies for at least two immunogenic proteins simultaneously.

10. Method according to claim 1 where measurement of the biomolecular interaction on the gradient for finding the fixed Rₘₐₓ value includes analysis of the on- and off rates using the sensorgrams with the fixed Rₘₐₓ value for all ligand gradients while simultaneously the concentration is measured at another location on the gradient.

11. Method according to claim 10 where the biomolecular interaction on the gradients are simultaneously exposed to concatenated injections of anti-isotype antibodies.

12. Method according to claim 1 where the strength of binding of isotypes of antibodies using SPR imaging and method to reduce the number of false positives characterized by a print of gradients of ligands exposed to patient samples.

13. (canceled)

14. (canceled)

15. (canceled)

16. (canceled)

17. Method according to claim 1 where the immune system related disease is COVID-19.
